(19) United States
(12) Patent Application Publication
(10) Pub. No.: US 2007/0025943 A1
(43) Pub. Date: Feb. 1, 2007

(54) Make-up compositions containing a film forming polyurethane and a film-forming (meth)acrylate copolymer

(75) Inventor: Lilavati Patel, Edison, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: L'Oreal S.A., Paris (FR)

(21) Appl. No.: 11/194,317

(22) Filed: Aug. 1, 2005

(51) Int. Cl. A61K 8/81

(52) U.S. Cl. 424/70.7; 424/70.16; 424/70.17

(57) ABSTRACT

Disclosed are cosmetic compositions comprising a film forming polyurethane and a film forming (meth)acrylate copolymer, and a cosmetically acceptable carrier, and methods of making and using them.

### BACKGROUND OF THE INVENTION

Film-formers are included in cosmetic formulations because they allow the formulation to adhere to keratin materials such as the skin or hair. Film-formers are important ingredients in many cosmetic compositions including mascaras and foundations.

The cosmetic industry focuses much of its efforts, in regards to mascara, on increasing two fundamental properties, namely enhancing volume or thickness of eyelashes and extending length of wear. U.S. Pat. No. 5,874,072 teaches mascara containing a mixture of water-insoluble copolymers in the form of an aqueous emulsion with water-soluble film-forming polymers. U.S. Pat. No. 6,248,336 teaches mascara compositions with improved wear characteristics in the form of an emulsion comprising an insoluble polymeric material in an aqueous emulsion, and lipophilic oil components including a polyvinylpyrrolidone hexadecane copolymer. There have also been advancements in creating an effect of eyelashes with increased volume. For example, U.S. Pat. No. 6,726,917 discloses a mascara containing fibers, pigments, and at least two film formers: at least one tacky film former soluble or dispersible in water and at least one tacky film former soluble in oil. Similarly, U.S. Pat. No. 6,503,521 teaches mascara that enhances volume via the use of three film farmers, namely: at least one tacky film former soluble or dispersible in water; at least one tacky film former soluble in oil; and at least one additional water-soluble or water-dispersible film former.

### SUMMARY OF THE INVENTION

A first aspect of the present invention is directed to a cosmetic composition comprising a cosmetically acceptable carrier, a film-forming polyurethane and a film-forming (meth)acrylate copolymer. The compositions may have at least one other cosmetically acceptable ingredient.

A second aspect of the present invention is directed to a method of preparing the cosmetic composition, which in some embodiments entails premixing the polyurethane and the (meth)acrylate copolymer prior to combining them with the other ingredient(s) of the composition.

A third aspect of the present invention is directed to a method of applying make-up to keratinous tissue, which comprises applying the cosmetic composition to the keratinous tissue (e.g., skin, hair or lips).

A fourth aspect of the present invention is directed to a method of lengthening and/or volumizing (or thickening) eyelashes, comprising applying to eyelashes a cosmetic composition comprising a cosmetically acceptable carrier, and a film-forming polyurethane and a film-forming (meth)acrylate copolymer.

Compositions containing these film-forming polymers provide several advantages. For example, in the case of mascaras, advantages include one or more of buildability, lengthening, high gloss, good wear characteristics and volumizing benefit.

### DETAILED DESCRIPTION

The present invention relates to a cosmetic composition comprising at least two film-forming polymers comprising a polyurethane and a copolymer of an acrylate, which is intended for making up or for cosmetic care of keratin tissue such as keratin fibres, for instance the eyelashes, the eyebrows or the hair, and also the skin (including lips, facial skin and body) and the nails, especially of human beings. The invention also relates to a cosmetic care and make-up process for keratin materials, as well as to methods for making the compositions.

The composition may be in the form of mascara, eyeliner (e.g., liquid eye liner), products for the lips, powders (e.g., face powder), eye shadows (e.g., liquid eye shadow and eye shadow bases), make-up products for the body, blush (e.g., liquid blush), concealers, foundations, products for the nails such as nail polish, topical hair coloring compositions (e.g., hair mascara) and hair styling compositions (e.g., gels, mousses, sprays, lacquers, pomades, and glossers), compositions for caring for or treating hair such as shampoos, conditioners, hair dyeing compositions, and hair bleaching, relaxing or permanent hair waving compositions, anti-sun compositions, skin-coloring compositions or skincare products such as moisturizing compositions or compositions containing a medicament.

More especially, the invention relates to make-up for eyes and lips such as mascara, eye liner, eye shadow and lip compositions. The term “mascara” as used herein refers to a composition intended to be applied to the eyelashes: it may be a make-up composition for the eyelashes, a make-up base for the eyelashes, a composition to be applied over a mascara, also known as a top coat, or even a cosmetic treatment composition for the eyelashes. The mascara is more particularly intended for human eyelashes, but also for false eyelashes.

The compositions of the invention may take a variety of forms, including a paste, a solid or a more or less viscous cream. Further, the inventive composition may be a single emulsion (such as an oil-in-water or water-in-oil emulsion), a multiple emulsion (such as an oil-in-water-in-oil emulsion or a water-in-oil-in-water emulsion), or a rigid or soft gel comprising an oily continuous phase. The compositions of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally comprising dispersed lipid vesicles and/or oil droplets, and a fatty phase optionally comprising dispersed lipid vesicles and/or water droplets. In some embodiments, the composition has a continuous oily phase or fatty phase and is more specifically an anhydrous composition, for example, a stick or dish form. An anhydrous composition is one that has less than 10% water by weight, such as, for example, less than 5% by weight, and which includes compositions that do not contain added water (i.e., water as a separate ingredient).

The film-forming polymers of the present invention have as one component, a film-forming polyurethane, which may be chosen from aliphatic, cycloaliphatic and aromatic polyurethanes, polyurea-urethane and polyurea copolymers, comprising at least one block chosen from:
at least one block chosen from aliphatic, cycloaliphatic and aromatic polyester origins,
at least one silicone block chosen from branched and non-branched silicone blocks, for example polydimethylsiloxane and polymethylphenylsiloxane, and
at least one block comprising at least one fluoro group.

The polyurethanes that may be used may also be obtained from branched or non-branched polyesters, or from alkyls comprising labile hydrogens that are modified by reaction with a diisocyanate and a difunctional organic compound (for example dihydro, diamino or hydroxyamino), and comprising either a carboxylic acid or carboxylate group, or a sulphonic acid or sulphonate group, or alternatively a neutralizable tertiary amine group or a quaternary ammonium group.

The polyurethane may be in the form of an aqueous disperson. Such polyurethanes include those manufactured or sold under the names Neorez R-981® and Neorez R-974® by the company Avecia-Neoresins, Avalure UR-405, Avalure UR-410®, Avalure UR-425® and Avalure UR-450® available from Noveon (Ohio), Sancure 875®, Sancure 861®, Sancure 878® and Sancure 2060® by the company Goodrich, and Impranil 85® by the company Bayer, and mixtures thereof. In preferred embodiments, the polyurethane is in the form of an aqueous disperson of the copolymer PPG-17/IPDI/DMPA (polypropylene glycol with n=17 units/isphorone diisocyanate/dimethylol propionic acid), commercially available from Noveon under the tradename Avalure UR-450®.

The other film-forming polymer is a copolymer of a (meth)acrylic acid and its esters, referred to herein as an (meth)acrylate copolymer. In preferred embodiments, the copolymer is an acrylates copolymer, which as used herein, refers to a copolymer of two or more monomers selected from the group consisting of acrylic acid, methacrylic acid, and their simple esters, e.g., lower alkyl esters such as methyl and ethyl esters. Such copolymers, which may be in the form of an aqueous dispersion, are commercially available from numerous sources, including Covacryl A15 and Covacryl E14 by Wackherr, Luviflex Soft from BASF, Luvimer 36D from BASF, Yodosol GH800 from National Starch and Carboset XL-40 from BF Goodrich. In preferred embodiments, the acrylates copolymer is an aqueous dispersion consisting of the ethyl ester of acrylic acid and the methyl ester of methacrylic acid, and which is commercially available from Daito Kasei under the tradename Daitosol 5000AD (CAS #2135-39-1, which is an aqueous emulsion having a solids content of about 50%).

Other film-forming (meth)acrylate copolymers are acrylates/ethylhexyl include but are not limited to acrylate copolymers (Daitosol 5000SJ by Daito Kasei), butyl acrylate/hydroxypropyl dimethicone acrylate copolymers (Granacrysil BAS by Grant Industries, Inc.), acrylates/C12-C22 alkylmethacrylate copolymers (Allianz OPT by ISP), isododecane and acrylates copolymers (Giovarez AC-5099M by Phoenix), and acrylates/octylacrylamide copolymers (Dermacryl-79 by National Starch & Chemical Company).

The total amount of the two film forming polymers generally ranges from about 0.5 to about 40% by dry weight of the composition, and preferably from about 2.0 to about 30% by dry weight of the composition. The ratio of the polyurethane to the acrylates copolymer generally varies from about 3:7 to about 7:3, and preferably from about 1:1 to about 3:2, based on dry weight of the polymers. In some cases, the ratio of the polyurethane to the acrylates copolymer may be higher than 7:3, e.g., 71:29, 72:28, 73:27, 74:26, 75:25, 76:24, 77:23, 78:22, 79:21, 80:20, and higher.

The compositions of the invention contain a cosmetically acceptable carrier, which may contain a fatty phase, a fatty and aqueous phase, or an aqueous phase. The fatty phase typically contains one or more of fatty substances chosen from oils, organic solvents, waxes, pasty fatty substances, and mixtures thereof. The fatty phase can form a continuous phase of the composition. In particular, the composition according to the invention may be anhydrous. The fatty phase may especially contain any oil which is physiologically acceptable and in particular cosmetically acceptable, chosen especially from oils of mineral, animal, plant or synthetic origin, carbon-based oils, hydrocarbon-based oils, fluoro oils and/or silicone oils, alone or as a mixture, provided that they form a homogeneous and stable mixture and provided that they are compatible with the intended use. The total fatty phase of the compositions of the present invention can represent from about 2% to about 98% by weight, relative to the total weight of the composition, and preferably from about 5% to about 85% by weight.

The composition according to the invention, e.g., in the case of lipsticks, may contain at least one fatty compound that is pasty at room temperature. For the purposes of the invention, the expression “pasty fatty substance” means fatty substances with a melting point ranging from 20 to 55° C., preferably 25 to 45° C., and/or a viscosity at 40° C. ranging from 0.1 to 40 Pa·s (1 to 400 Pascal seconds), preferably 0.5 to 25 Pa·s, measured using a Contraves TV or Rhomat 80 viscometer, equipped with a spindle rotating at 60 Hz. A person skilled in the art can select the spindle for measuring the viscosity from the spindles MS-r3 and MS-r4, on the basis of his general knowledge, so as to be able to carry out the measurement of the pasty compound tested.

These fatty substances are preferably hydrocarbon-based compounds, optionally of polymeric type; they can also be chosen from silicone compounds and/or fluoro compounds; they may also be in the form of a mixture of hydrocarbon-based compounds and/or silicone compounds and/or fluoro compounds. In the case of a mixture of different pasty fatty substances, the hydrocarbon-based pasty compounds (containing mainly hydrogen and carbon atoms and optionally ester groups) are preferably used in major proportion.

Among the pasty compounds which may be used in the composition according to the invention, mention may be made of lanolins and lanolin derivatives such as acetylated lanolins or oxypropylenated lanolins or isopropyl lanolate, having a viscosity of from 18 to 21 Pa·s, preferably 19 to 20.5 Pa·s, and/or a melting point of from 30 to 55° C., and mixtures thereof. It is also possible to use esters of fatty acids or of fatty alcohols, in particular those containing from 20 to 65 carbon atoms (melting point of about from 20 to 35° C. and/or viscosity at 40° C. ranging from 0.1 to 40 Pa·s), such as triisostearyl or cetyl citrate; arachidyl propionate; polyvinyl laurate; cholesterol esters, such as triglycerides of plant origin, such as hydrogenated plant oils, viscous polyesters such as poly(12-hydroxystearic acid), and mixtures thereof. Triglycerides of plant origin which may be used are hydrogenated castor oil derivatives, such as “Thixinr” from Rhox.

Mention may also be made of pasty silicone fatty substances such as polydimethylsiloxanes (PDMSs) containing pendent chains of the alkyl or alkoxy type containing from 8 to 24 carbon atoms, and having a melting point of 20-55° C., such as stearyldimethicones, in particular those sold by Dow Corning under the trade names DC2503 and DC25514, and mixtures thereof.

The pasty fatty substance may be present in the compositions according to the invention in a proportion of from 0% to 60% (in particular 0.01% to 60%) by weight, preferably in a proportion of from 0.5% to 45% by weight, and more preferably from 2% to 30% by weight of the composition.

The fatty phase of the composition according to the invention can comprise a wax. For the purposes of the present invention, the term “wax” means a lipophilic fatty compound that is solid at room temperature (25° C.) and atmospheric pressure (760 mm Hg, i.e. 10⁵ Pa), which undergoes a reversible solid/liquid change of state and which has a melting point of greater than 30° C., or greater than 55° C., which may be up to 200° C., in particular up to 120° C.

By taking the wax to its melting point, it is possible to make it miscible with oils and to form a microscopically homogeneous mixture, but on returning the temperature of the mixture to room temperature, recrystallization of the wax in the mixture of oils is obtained. According to the invention, the melting point values correspond to the melting peak measured using a differential scanning calorimeter (DSC), for example the calorimeter sold under the name DSC 30 by the company Mettler, with a temperature increase of 5 or 10° C. per minute.

For the purposes of the invention, the waxes are those generally used in cosmetics and dermatology. A variety of waxes may be useful, including waxes of animal origin, waxes of plant origin, waxes of mineral origin and waxes of synthetic origin. Examples of waxes of animal origin include but are not limited to beeswaxes, lanolin waxes and Chinese insect waxes. Examples of waxes of plant origin include rice waxes, carnauba wax, candellila wax and ouricurry wax, cork fibre waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes, waxes obtained by Fischer-Tropsch synthesis, waxy copolymers and their esters, and silicone and fluoro waxes. Alternatively, hydrogenated oils of animal or plant origin may be used. Examples include hydrogenated jojoba waxes and hydrogenated oils which are obtained by catalytic hydrogenation of fats composed of a C₈-C₃₂ linear or nonlinear fatty chain, hydrogenated sunflower oil, hydrogenated castor oil, hydrogenated copra oil, hydrogenated lanolin and hydrogenated palm oils.

The waxes present in the composition may be dispersed in the form of particles in an aqueous medium. These particles may have an average size ranging from 50 nm to 10 μm and preferably from 50 nm to 3.5 μm. In particular, the wax may be present in the form of a wax-in-water emulsion, the waxes possibly being in the form of particles with an average size ranging from 1 μm to 10 μm and preferably from 1 μm to 3.5 μm. In another embodiment of the composition according to the invention, the wax may be present in the form of a wax microdispersion, the wax being in the form of particles with an average size of less than 1 μm and in particular ranging from 50 nm to 500 nm. Wax microdispersions are disclosed in EP-A-557 196 and EP-A-1 048 282.

The wax may also have a hardness ranging from 0.05 MPa to 15 MPa and preferably ranging from 6 MPa to 15 MPa. The hardness is determined by measuring the compressive strength, measured at 20° C. using a texturometer sold under the name TA-XT2i by the company Rheo, equipped with a stainless steel cylinder 2 mm in diameter travelling at a measuring speed of 0.1 mm/s, and penetrating into the wax to a penetration depth of 0.3 mm. To carry out the hardness measurement, the wax is melted at a temperature equal to the melting point of the wax +20° C. The molten wax is cast in a container 30 mm in diameter and 20 mm deep. The wax is recrystallized at room temperature (25° C.) over 24 hours and is then stored for at least one hour at 20° C. before carrying out the hardness measurement. The value of the hardness is the compressive strength measured divided by the area of the texturometer cylinder in contact with the wax.

The wax may be present in the composition according to the invention in an amount generally ranging from about 0.1% to about 50% by weight, relative to the total weight of the composition, preferably from about 0.5% to about 30% by weight, and more preferably from about 1% to about 20% by weight.

As disclosed herein, the term “liquid fatty phase” means a fatty phase that is liquid at room temperature (25° C.) and atmospheric pressure (760 mmHg, i.e., 105 Pa), comprising at least one-fatty substance that is liquid at room temperature, also known as oils, which are generally mutually compatible.

For instance, the oils may be chosen from any physiologically acceptable oils, such as cosmetically acceptable oils, for example, mineral, animal, plant or synthetic oils; further for example, oils chosen from volatile and non-volatile, hydrocarbon-based oils, silicone oils, and fluoro oils, and mixtures thereof. The term “hydrocarbon-based oil” means an oil mainly comprising carbon and hydrogen atoms and possibly at least one functional group chosen from hydroxyl, ester, ether and carboxylic functional groups. For example, the oils may have a viscosity ranging from 0.5 to 300,000 cps, further for example, from 50 to 50,000 cps, and even further for example, from 100 to 100,000 cps.

Among non-limiting examples of oils that may be used, mention may be made of:
hydrocarbon-based oils of animal origin, such as perhydrosqualene;
hydrocarbon-based plant oils such as liquid triglycerides of fatty acids of from 4 to 24 carbon atoms, for instance heptanoic or octanoic acid triglyceride, or alternatively sunflower oil, maize oil, soybean oil, marrow oil, grapeseed oil, sesame seed oil, hazelnut oil, apricot oil, macadamia oil, castor oil, avocado oil, caprylic/capric acid triglycerides, for instance those sold by the company Stearineries Dubois or those sold under the names Miglyol 810, 812 and 818 by the company Dynamit Nobel, jojoba oil and shea butter;
linear and branched hydrocarbons of mineral or synthetic origin, for example, liquid paraffin and derivatives thereof, petroleum jelly, polydecenes, polybutenes and hydrogenated polyisobutene, for example parleam;
synthetic esters and ethers, such as those of fatty acids, for instance the oils of formula R4COOR5 wherein R4 is chosen from higher fatty acid residues comprising from 1 to 40 carbon atoms and R₅ is chosen from hydrocarbon-based chains comprising from 1 to 40 carbon atoms with the number of carbon atoms in (R₄+R₅) being equal or greater than 10, for instance purcellin oil, isononyl isononanoate, isopropyl myristate, 2-ethylhexyl palmitate, 2-octyldodecyl stearate, 2-octyldodecyl erucate, isostearyl isostearate and tridecyl trimellitate; hydroxylated esters, for instance isostearyl lactate, octyl hydroxystearate, octyldodecyl hydroxystearate, diisostearyl malate, triisocetyl citrate and fatty alkyl heptanoates, octanoates and decanoates; polyol esters, for instance propylene glycol dioctanoate, neopentyl glycol diheptanoate and diethylene glycol diisononanoate; and pentaerythritol esters, for instance pentaerythrityl tetraisostearate;
fatty alcohols comprising from 12 to 26 carbon atoms, for instance octyldodecanol, 2-butyloctanol, 2-hexyldecanol 2-undecylpentadecanol and oleyl alcohol;
fluoro oils chosen from optionally partially hydrocarbon-based and silicone-based fluoro oils;

silicone oils, chosen, for example, from volatile and non-volatile, linear and cyclic polydimethylsiloxanes (PDMSs); polydimethylsiloxanes comprising at least one group chosen from alkyl, alkoxy and phenyl groups, which is pendent or at the end of a silicone chain, wherein the at least one group comprises from 2 to 24 carbon atoms; phenyl silicones, for instance phenyl trimethicones, phenyl dimethicones, phenyl trimethylsiloxy diphenyl siloxanes, diphenyl dimethicones, diphenyl methyldiphenyl trisiloxanes and 2-phenyl ethyl trimethyl siloxysilicates, and mixtures thereof.

In some embodiments, the cosmetically acceptable carrier contains an aqueous medium, constituting an aqueous phase, with or without a fatty phase. The aqueous phase may be the continuous phase of the composition. The aqueous phase may consist essentially of water; it may also comprise a mixture of water and of water-miscible solvent (miscibility in water of greater than 50% by weight at 25° C.), for instance, lower monoalcohols containing from 1 to 5 carbon atoms such as ethanol or isopropanol, glycols containing from 2 to 8 carbon atoms, such as propylene glycol, ethylene glycol, butylene glycol or dipropylene glycol, C3-C4 ketones and C2-C4 aldehydes. The aqueous phase (water and optionally the water-miscible organic solvent) may be present in a content ranging from about 1% to about 95% by weight, relative to the total weight of the composition, preferably from about 3% to about 80% by weight, and more preferably from about 5% to about 60% by weight.

The compositions of the invention may contain surfactants. Surfactants typically employed in the compositions of the present invention include amphoteric, anionic, cationic and nonionic surfactants. See, e.g., Encyclopedia of Chemical Technology, KIRK-OTHMER, volume 22, pp. 333-432, 3rd edition, 1979, Wiley, for the definition of the properties and (emulsifying) functions of the surfactants, in particular pp. 347-377 of this publication regarding anionic and nonionic surfactants. Examples of surfactants useful in the compositions of the invention include but are not limited to as nonionic surfactants, fatty acids, fatty alcohols, polyethoxylated fatty alcohols or polyglycerolated fatty alcohols, such as polyethoxylated stearyl alcohols or cetylstearyl alcohols, esters of fatty acid and sucrose, and glucose alkyl esters, in particular polyoxyethylenated C₁-C₆ alkyl glucose fatty esters, and as anionic surfactants, C₁₆-C₃₀ fatty acids neutralized by amines, ammonia or the alkali metal salts thereof. Examples of amphoteric surfactants include but are not limited to betaines, sultaines, hydroxysultaines, alkyl amphodiacetates, alkyl amphodipropionates, and imidazolines, or salts thereof. Other fatty acid condensates such as those formed with amino acids, proteins, and the like are suitable as well. Specific examples include cocamphodipropionate, e.g., Miranol C2M-SF (disodium cocamphodipropionate), in its salt-free form, available from Rhone-Poulenc, and Crosultaine C-50 (cocamidopropyl hydroxysultaine), available from Croda. Examples of cationic surfactants include but are not limited to quaternary amines, amine oxides and amines, e.g., alkyl amines, alkyl imidazolines, ethoxylated amines, quaternary compounds, and quaternized esters.

In certain embodiments e.g., wherein the composition is in the form of washable mascara, surfactants are selected in order to obtain an oil-in-water emulsion. In some embodiments, the surfactants include triethanolamine and stearic acid. In certain other embodiments, e.g., wherein the composition is in the form of waterproof mascara, surfactants are often used to facilitate dispersion of pigments. Preferred examples include lecithins. Lecithins are mixtures of phospholipids, i.e., of diglycerides of fatty acids linked to an ester of phosphoric acid. Preferably, lecithins are diglycerides of stearic, palmitic, and oleic acids linked to the choline ester of phosphoric acid. Lecithin is usually defined either as pure phosphatidyl cholines or as crude mixtures of phospholipids that include phosphatidyl choline, phosphatidyl serine, phosphatidyl ethanolamine, phosphatidyl inositol, other phospholipids, and a variety of other compounds such as fatty acids, triglycerides, sterols, carbohydrates, and glycolipids. See, e.g., U.S. Pat. Nos. 6,015,574 and 6,221,389. Lecithins may also be present in washable mascaras.

Surfactants are generally present in amounts ranging from about 2 to about 30% by weight, and preferably from about 5% to about 15% by weight, relative to the total weight of the composition.

Depending upon the nature of the inventive composition, controlling viscosity may be important from the standpoints of fast and easy application of the composition, as well as uniform coating. In the case of washable and waterproof mascaras, for example, viscosity of washable mascaras generally ranges from about 10 to about 60 pascal seconds (Pa·s), and preferably from about 20 to about 40 Pa·s, whereas viscosity of waterproof mascaras generally ranges from about 10 to about 70 Pa·s, and preferably from about 10 to about 40 Pa·s. Viscosity is measured at 25° C. with a Rheomat RM 180 viscometer fitted with a No. 4 rotor, wherein the measurement is carried out after spinning the rotor for 10 minutes (after which time stabilization of the viscosity and of the rotor spin speed are observed), at a shear rate of 200 s⁻¹.

Viscosity may be adjusted by adding a thickener. Representative examples include cellulose-based thickeners, for example, water-soluble cellulose-based thickeners, such as hydroxyethylcellulose, methylcellulose, hydroxypropylcellulose and carboxymethylcellulose. Among these thickeners, specific examples include alginates, maltodextrin, polysaccharide resins such as starch and its derivatives, hyaluronic acid and its salts, clays, and, in particular, montmorillonites, hectorites and laponites, crosslinked polyacrylic acids, such as the “Carbopol” products from the company Goodrich, the polyglyceryl(meth)acrylate polymers sold under the names “Hispagel” or “Lubragel” by the companies Hispano Quimica or Guardian, polyvinylpyrrolidone (PVP), polyvinyl alcohol, crosslinked acrylamide polymers and copolymers, such as those sold under the names “PAS 5161” or “Bozepol C” by the company Hoechst, “Sepigel 305” by the company SEPPIC, crosslinked methacryloyloxyethyltrimethylammonium chloride homopolymers sold under the name “Salcare SC95” by the company Allied Colloid, and associative polymers and, in particular, associative polyurethanes. Oil soluble thickening agents may also be used. See, U.S. Patent Publications 2003/0215413, 2005/0065046 and 2002/0028226.

The thickening agent is generally present in an amount ranging from about 0.05% to about 20% by weight, and preferably from about 0.5% to about 10% by weight.

Compositions of the present invention may contain a colorant. Colorants according to the present invention include but are not limited to lipophilic dyes, hydrophilic dyes, traditional pigments, and nacres usually used in cosmetic or dermatological compositions, and mixtures thereof. The colorant may have any shape, such as, for example, spheroidal, oval, platelet, irregular, and mixtures thereof. Pigments may optionally be surface-treated e.g., with silicones, perfluorinated compounds, lecithin, and amino acids.

The liposoluble dyes include, for example, Sudan Red, D&C Red 17, D&C Green 6, β-carotene, soybean oil, Sudan Brown, D&C Yellow 11, D&C Violet 2, D&C Orange 5, quinoline yellow and annatto. The water-soluble dyes are, for example, beetroot juice or methylene blue.

The pigments may be chosen from white pigments, colored pigments, inorganic pigments, organic pigments, coated pigments, uncoated pigments, pigments having a micron size and pigments not having a micron size. Among the inorganic pigments that may be mentioned are titanium dioxide, optionally surface-treated, zirconium oxide, zinc oxide, cerium oxide, chromium oxide, manganese violet, ultramarine blue, chromium hydrate, and ferric blue. Among the organic pigments which may be mentioned are carbon black, pigments of D&C type, lakes based on cochineal carmine, lakes based on barium, lakes based on strontium, lakes based on calcium, and lakes based on aluminium.

The nacreous pigments may, for example, be chosen from white nacreous pigments such as mica coated with titanium and mica coated with bismuth oxychloride, colored nacreous pigments such as titanium mica with iron oxides, titanium mica with, for example, ferric blue and/or chromium oxide, titanium mica with an organic pigment of the type mentioned above, as well as nacreous pigments based on bismuth oxychloride, interferential pigments, and goniochromatic pigments.

Colorants can generally be present in an amount ranging from about 0.01% to about 50% relative to the total weight of the composition.

The compositions of the present invention may also contain dispersion enhancing agents such as polysaccharide resins, e.g., KM 13, available from KAMA International Corp. (Duluth, Ga.). Dispersion enhancing agents are especially preferred in pigmented products.

Fillers and mothers-of-pearl may also be added to the formulations to modify the texture of the composition and the matteness/gloss effect. Fillers should be understood to mean lamellar or non-lamellar, inorganic or synthetic, colorless or white particles. Mothers-of-pearl should be understood to mean irridescent particles produced especially by certain mollusks in their shell or else synthesized. Pearling agents that may be used in the practice of the invention include mica, iron oxides, titanium dioxide and any other pearling agent known in the cosmetic arts.

The compositions may further comprise one or more fillers. As used herein, the term “filler” means any particle that is solid at room temperature and atmospheric pressure, used alone or in combination, which does not react chemically with the various ingredients of the emulsion and which is insoluble in these ingredients, even when these ingredients are raised to a temperature above room temperature and in particular to their softening point or their melting point. The filler may be absorbent, i.e., capable in particular of absorbing the oils of the composition and also the biological substances secreted by the skin. The filler may be surface-treated, e.g., with lecithin, silicones, amino acids, fatty acids, fatty alcohols, or metallic soaps e.g., to make it lipophilic, and/or may be porous so as to absorb the sweat and/or sebum secreted by the skin.

The filler, e.g., powders, may be chosen from inorganic and organic fillers, and may have any shape such as lamellar, spherical and/or oblong. Examples of fillers include talc, mica, fumed silica, kaolin, poly-β-alanine powders, acrylic polymer powders (such as acrylic acid copolymer powder sold by Dow Corning as Polytrap®), lauroyllysine, bismuth oxychloride, starch, starch derivatives, hollow polymer microspheres (such as those hollow polymer microspheres formed from polyvinylidene chloride and acrylonitrile, for instance the product sold by Nobel Industrie as Expancel®), and polymerized silicone microspheres (such as those polymerized silicone microspheres sold by Toshiba as Tospearl®), precipitated calcium carbonate, magnesium carbonate and hydrocarbonate, hydroxyapatite, ceramic microcapsules, polyester particles and coated elastomers such as products sold under the denomination KSP (KSP100, KSP 200, KSP 300) sold by Shin Etsu and/or those described in U.S. Pat. No. 5,538,793.

Filler(s) may be present in an amount ranging from about 0.1% to about 25%, for example, from about 1% to about 20% by weight of the total weight of the composition.

In some embodiments, such as mascaras, the composition further comprise fibers to allow an improvement in the lengthening effect.

The fibers useful in the present invention may be chosen from natural and synthetic fibers. Natural fibers include, but are not limited to, cotton, silk, wool, and other keratin fibers. Synthetic fibers include, but are not limited to, polyester, rayon, nylon and other polyamide fibers.

Yet other fibers useful in the present invention include those described in EP 1172078. The fibers disclosed in this publication include types of elastofibers. These fibers are chemical fibers, extremely stretchable, and which regain their primary shape as soon as the tractive force is interrupted. Representative examples include elastane (abbreviations: EL or Spandex®)), highly polymerized fibers, which contain at least 85% by weight of segmented polyurethane, and elastodiene fibers (abbreviation: ED) containing synthetic polyisoprenes or high polymers, which are obtained from the polymerization of one or more dienes, by optionally adding one or several vinyl monomers. Rubbery fibers (abbreviation: LA) issued from natural rubber may also be included in the second group. The elastodienes are often vulcanized. A fiber composed of both polyamide and polyurethane also has elastic properties.

The fibers are present in an amount generally ranging from about 0.1% to about 20% relative to the total weight of the composition. In some embodiments, the fibers are present in an amount ranging from about 0.2% to about 10% relative to the total weight of the composition. The fibers typically have an average length ranging from about 0.5 mm to about 4.0 mm, such as from about 1.5 mm to about 2.5 mm.

Compositions of the present invention may further contain a sunscreen agent. Sunscreens useful in the present invention include organic sunscreens and/or inorganic sunscreens which are preferably active in the UV-A and/or UV-B regions (absorbers), and are soluble in water or in fats or insoluble in, e.g., cosmetic solvents commonly used. The sunscreens useful in the present invention preferably comprise chemical absorbers. Typically, the compositions of the present invention contain combinations of two or more sunscreens.

Organic sunscreens which are useful herein include ethylhexyl salicylate, ethylhexyl methoxycinnamate, octocrylene, phenylbenzimidazole sulphonic acid, benzophenone-3, benzophenone-4, benzophenone-5,4-methylbenzylidene camphor, terephthalylidene dicamphor sulphonic acid, disodium phenyl dibenzimidazole tetrasulphonate, 2,4,6-tris(diisobutyl 4′-aminobenzalmalonate)-s-triazine, anisotriazine, ethylhexyl triazone, diethylhexyl butamido triazone, methylene bisbenzotriazolyl tetramethylbutylphenol, drometrizole trisiloxane, 1,1′-dicarboxy(2,2′-dimethylpropyl)-4,4-diphenylbutadiene, and their mixtures.

Representative inorganic sunscreens include pigments or alternatively nanopigments (mean size of the primary particles: generally between 5 nm and 100 nm, preferably between 10 nm and 50 nm) formed from coated or uncoated metal oxides, such as, for example, titanium oxide (amorphous or crystalline in the rutile and/or anatase form), iron oxide, zinc oxide, zirconium oxide or cerium oxide nanopigments, which are all known in the art as UV sunscreens. Conventional coating agents are, furthermore, alumina and/or aluminium stearate. Such nanopigments formed from coated or uncoated metal oxides are disclosed in particular in EP 518 772 and EP 518 773.

Another group of sunscreens useful in the present invention are certain UV-A and UV-B absorbers. Classifying the chemical absorbers generally as UV-A or UV-B absorbers is accepted within the industry. However, a more precise classification is one based upon the chemical properties of the sunscreens. There are eight major classifications of sunscreen chemical properties that are discussed at length in “Sunscreens—Development, Evaluation and Regulatory Aspects,” by N. Shaath, et al., 2nd. Edition, pages 269-273, Marcel Dekker, Inc. (1997). This discussion, in its entirety, is incorporated by reference herein.

Preferred UV-A absorbers generally absorb radiation in the 320 to 400 nm region of the ultraviolet spectrum. Such preferred UV-A absorbers include anthranilates, benzophenones, and dibenzoyl methanes. Preferred UV-B absorbers generally absorb radiation in the 280 to 320 nm region of the ultraviolet spectrum. Such preferred UV-B absorbers include p-aminobenzoic acid derivatives, camphor derivatives, cinnamates, and salicylates. Other sunscreens active in the UV-A and/or UV-B range include:
p-aminobenzoic acid;
oxyethylene (25 mol) p-aminobenzoate;
2-ethylhexyl p-dimethylaminobenzoate;
ethyl N-oxypropylene p-aminobenzoate;
glycerol p-aminobenzoate;
4-isopropylbenzyl salicylate;
2-ethylhexyl 4-methoxycinnamate;
methyl diisopropylcinnamate;
isoamyl 4-methoxycinnamate;
diethanolamine 4-methoxycinnamate;
3-(4′-trimethylammunium)-benzyliden-bornan-2-one methylsulfate;
2-hydroxy-4-methoxybenzophenone;
2-hydroxy-4-methoxybenzophenone-5-sulfonate;
2,4-dihydroxybenzophenone;
2,2′,4,4-tetrahydroxybenzophenone;
2,2′-dihydroxy-4,4′dimethoxybenzophenone;
2-hydroxy-4-n-octoxybenzophenone;
2-hydroxy-4-methoxy-4′-methoxybenzophenone;
(2-oxoborn-3-ylidene)-tolyl-4-sulfonic acid and soluble salts thereof;
3-(4′-sulfo)benzyliden-bornan-2-one and soluble salts thereof;
3-(4′methylbenzylidene)-d,l-camphor;
3-benzylidene-d,l-camphor;
benzene 1,4-di(3-methylidene-10-camphosulfonic) acid and salts thereof (the product Mexoryl SX described in U.S. Pat. No. 4,585,597);
urocanic acid;
2,4,6-tris[p-(2′-ethylhexyl-1′-oxycarbonyl)-anilino]-1,3,5-triazine-,
2-[(p-(tertiobutylamido)anilino]-4,6-bis-[(p-(2′-ethylhexyl-1′-oxycarbonyl)anilino]-1,3,5-triazine;
2,4-bis {[4-(2-ethyl-hexyloxy)]-2-hydroxyl-phenyl}-6-(4-methoxy-phenyl)-1,3,5-triazine (“TINOSORB S” marketed by Ciba);
the polymer of N-(2,4)-[(2-oxoborn-3-yliden)methyl]benzyl]-acryl-amide;
1,4-bisbenzimidazolyl-phenylen-3,3′,5,5′-tetrasulfonic acid and salts thereof;
the benzalmalonate-substituted polyorganosiloxanes;
the benzotriazole-substituted polyorganosiloxanes (Drometrizole Trisiloxane);
dispersed 2,2′-methylene-bis-[6-(2H-benzotriazol-2-yl)-4-(1,1,3,3-tetramethylbutyl)phenol] such as that marketed under the trademark MIXXIM BB/100 by Fairmount Chemical, or micronized in dispersed form thereof such as that marketed under the trademark TINOSORB M by Ciba-Geigy; and
solubilized 2,2′-methylene-bis-[6-(2H-benzotriazol-2-yl)-4-(methyl)-phenol] such as that marketed under the trademark MIXXIM BB/200 by Fairmount Chemical.

The dibenzoyl methane derivatives other than avobenzone are described, for example, in FR-2,326,405, FR-2,440,933 and EP-0,114,607, hereby expressly incorporated by reference.

Other dibenzoyl methane sunscreens other than avobenzone include (whether singly or in any combination):
2-methyldibenzoylmethane;
4-methyldibenzoylmethane;
4-isopropyldibenzoylmethane;
4-tert.-butyldibenzoylmethane;
2,4-dimethyldibenzoylmethane;
2,5-dimethyldibenzoylmethane;
4,4′-diisopropyldibenzoylmethane;
4,4′-dimethoxydibenzoylmethane;
2-methyl-5-isopropyl-4′-methoxydibenzoylmethane;
2-methyl-5-tert-butyl-4′-methoxydibenzoylmethane;
2,4-dimethyl-4′-methoxydibenzoylmethane; and
2,6-dimethyl-4-tert-butyl-4′-methoxydibenzoylmethane.

Particularly useful sunscreens which may be formulated into the compositions of the present invention include chemical absorbers such as p-aminobenzoic acid derivatives, anthranilates, benzophenones, camphor derivatives, cinnamic derivatives, dibenzoyl methanes (such as avobenzone also known as Parsol® 1789), diphenylacrylate derivatives, salicylic derivatives, triazine derivatives, benzimidazole compounds, bis-benzoazolyl derivatives, methylene bis-(hydroxyphenylbenzotriazole) compounds, the sunscreen polymers and silicones, or mixtures thereof. These are variously described in U.S. Pat. Nos. 2,463,264, 4,367,390, 5,166,355 and 5,237,071 and in EP-0,863,145, EP-0,517,104, EP-0,570,838, EP-0,796,851, EP-0,775,698, EP-0,878,469, EP-0,933,376, EP-0,893,119, EP-0,669,323, GB-2,303,549, DE-1,972,184 and WO-93/04665, all of which are also expressly incorporated herein by reference. Also exemplary of the sunscreens which may be formulated into the compositions of this invention are physical blockers such as cerium oxides, chromium oxides, cobalt oxides, iron oxides, red petrolatum, silicone-treated titanium dioxide, titanium dioxide, zinc oxide, and/or zirconium oxide, or mixtures thereof.

A wide variety of sunscreens, all of which are useful herein, are further described in U.S. Pat. Nos. 5,087,445 and 5,073,372; and Chapter VIII of Cosmetics and Science and Technology, by Segarin, et al., pages 189 et seq. (1957), all of which are incorporated herein by reference in their entirety.

Additional sunscreens that can be used herein are described in pages 2954-2955 of the International Cosmetic Ingredient Dictionary and Handbook (9th ed. 2002).

Preferred sunscreens are organic sunscreen agents. In particular, the use of monomeric fluorinated alkyl ether in a sunscreen composition containing the active sunscreen agent octocrylene is particularly advantageous.

Typically, the at least one sunscreen agent is present in an amount ranging from about 0.01 to about 30% of the total weight of the composition.

The compositions of the present invention may comprise a conditioning agent including but not limited to humectants, moisturizers, or skin conditioners. A variety of these materials can be employed and each can be present at a level of from about 0.01% to about 20. These materials include, but are not limited to, guanidine; urea; glycolic acid and glycolate salts (e.g. ammonium and quaternary alkyl ammonium); salicylic acid; lactic acid and lactate salts (e.g., ammonium and quaternary alkyl ammonium); aloe vera in any of its variety of forms (e.g., aloe vera gel); polyhydroxy alcohols such as sorbitol, glycerol, hexanetriol, butanetriol, propylene glycol, butylene glycol, hexylene glycol and the like; polyethylene glycols; sugars (e.g., melibiose) and starches; sugar and starch derivatives (e.g., alkoxylated glucose, fructose); hyaluronic acid; lactamide monoethanolamine; acetamide monoethanolamine; and mixtures thereof.

Representative examples of preservatives include alkyl para-hydroxybenzoates, wherein the alkyl radical has from 1, 2, 3, 4, 5 or 6 carbon atoms and preferably from 1 to 4 carbon atoms e.g., methyl para-hydroxybenzoate (methylparaben), ethyl para-hydroxybenzoate (ethylparaben), propyl para-hydroxybenzoate (propylparaben), butyl para-hydroxybenzoate (butylparaben) and isobutyl para-hydroxybenzoate (isobutylparaben), and phenoxyethanol. Mixtures of preservatives may certainly be used, e.g., the mixture of methyl-paraben, ethylparaben, propylparaben and butylparaben sold under the name Nipastat by Nipa, and the mixture of phenoxyethanol, methylparaben, ethylparaben, propylparaben and butylparaben sold under the name Phenonip, also by Nipa. These preservatives may be present in amounts ranging from about 0.01% to about 15% by weight and preferably from 0.5% to about 10% by weight relative to the total weight of the composition.

Plasticizers may also be added to the compositions to improve the flexibility and cosmetic properties of the resulting formulation. Plasticizers are materials which soften synthetic polymers. They are frequently required to avoid brittleness and cracking of film formers. One skilled in the art may routinely vary the amount of plasticizer desired based on the properties desired and the application envisaged. Plasticizers useful in the practice of the invention include lecithin, polysorbates, dimethicone copolyol, glycols, citrate esters, glycerin, dimethicone, and other similar ingredients disclosed in the International Cosmetic Dictionary and Handbook, Vol. 2 (7th ed. 1997), more particularly the plasticizers disclosed on page 1654.

The compositions of the present invention may further comprise at least one suitable (e.g., cosmetically or dermatologically acceptable) additive or adjuvant, including, for example, chelators such as EDTA and salts thereof, antioxidants (e.g., BHT, tocopherol), essential oils, fragrances, neutralizing agents, polymers, e.g., lipo-soluble polymers, and cosmetically active agents and dermatological active agents such as, for example, anti-inflammatory agents, defoaming agents, vitamins e.g., vitamins E and A, trace elements and essential fatty acids.

The present invention is further described in terms of the following non-limiting example(s). Unless otherwise indicated, all parts and percentages are on a weight-by-weight basis.

### EXAMPLES

### Example 1

### Mascara

| | CFTA NAME | % |
|---|---|---|
| A | Water | 39.200 |
| | Butylene Glycol | 3.000 |
| | Hydroxyethylcellulose | 0.350 |
| | Methylparaben | 0.250 |
| | Disodium EDTA | 0.200 |
| | Triethanolamine | 1.500 |
| | Simethicone | 0.100 |
| | Polysaccharide Resin | 1.000 |
| | Black Iron Oxide | 8.000 |
| B | Waxes | 15.500 |
| | Glyceryl Stearate | 4.000 |
| | Stearic Acid | 3.000 |
| | Butylparaben | 0.100 |
| C | Cyclopentasiloxane/Dimethiconol | 3.000 |
| | Dimethicone Copolyol | 0.200 |
| D | PPG-17/IPDI/DMPA/Copolymer | 10.000 |
| | Acrylates Copolymer | 10.000 |
| E | Phenoxyethanol | 0.500 |
| | | 100.000 |

To prepare the mascara, the water was heated to 60° C. in a beaker. Premixed butylene glycol and hydroxyethylcellulose were added to the water. Upon dispersion, methylparaben, disodium EDTA, triethanolanime, simethicone, and polysaccharide resin respectively, were added with mixing. Upon achieving a uniform dispersion, iron oxide was added and the resultant Phase A was milled until the pigment was well dispersed. The resultant dispersion was transferred to a paddle mixer. Phase A was heated to 85° C. to 90° C. Phase B ingredients were mixed separately and then heated to 85° C. to 90° C. Phase B was added to phase A, followed by emulsifying for 15 minutes using a Greerco® homogenizer. After emulsification was complete, the mixture was transferred to a sweep mixer and air-cooled to 60° C. Premixed phase C was added slowly. Once uniform, premade-premixed phase D was added slowly at 55° C. Once uniform, phase E was added very slowly, at a temperature of 45° C., and mixed until uniform. The resultant mixture was cooled to 30-32° C.

All publications cited in the specification are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A cosmetic composition comprising a film forming polyurethane and a film forming (meth)acrylate copolymer, and a cosmetically acceptable carrier.

2. The composition of claim 1, wherein the (meth)acrylate copolymer comprises a lower alkyl ester of acrylic acid and a lower alkyl ester of methacrylic acid.

3. The composition of claim 1, wherein the (meth)acrylate copolymer comprises ethyl acrylate and methyl methacrylate.

4. The composition of claim 1, wherein the polyurethane comprises PPG-17/IPDI/DMPA.

5. The composition of claim 1, wherein the (meth)acrylate copolymer comprises ethyl acrylate and methyl methacrylate, and wherein the polyurethane comprises PPG-17/IPDI/DMPA.

6. The composition of claim 1, wherein the polyurethane film forming polymer and the (meth)acrylate copolymer are present in a ratio of about 3:4 to about 3:2 based on dry weight.

7. The composition of claim 1, wherein the polyurethane film forming polymer and the (meth)acrylate copolymer together are present in an amount of about 0.5 to about 40% by dry weight of said composition.

8. The composition of claim 1, wherein the cosmetically acceptable carrier comprises a fatty phase comprising a pasty substance, a wax and/or oil.

9. The composition of claim 8, wherein the fatty phase comprises at least one wax selected from the group consisting of beeswax, paraffin, carnauba wax, ozokerite, microcrystalline wax, synthetic wax and Jojoba butter.

10. The composition of claim 1, further comprising a silicone oil.

11. The composition of claim 1, which is anhydrous.

12. The composition of claim 1, wherein said carrier comprises an aqueous phase.

13. The composition of claim 1, further comprising an emulsifier.

14. The composition of claim 1, further comprising a thickening or gelling agent.

15. The composition of claim 1, further comprising a colorant.

16. The composition of claim 1, further comprising a filler.

17. The composition of claim 1, which is mascara.

18. A method of preparing a cosmetic composition, comprising forming a film forming polymer blend comprising a film-forming polyurethane and a film-forming (meth)acrylate copolymer, and combining the mixture with a cosmetically acceptable carrier.

19. A method of applying make-up to keratinous tissue, comprising applying to keratinous tissue a cosmetic composition comprising two film-forming polymers comprising a polyurethane and a (meth)acrylate copolymer, and a cosmetically acceptable carrier.

20. A method of lengthening and/or volumizing eyelashes, comprising applying to eyelashes a cosmetic composition comprising a film-forming polyurethane and a film-forming (meth)acrylate copolymer, and a cosmetically acceptable carrier.
